(19) United States
(12) Patent Application Publication
(10) Pub. No.: US 2010/0267635 A1
(43) Pub. Date: Oct. 21, 2010

(54) USE OF PROTEASE INHIBITORS AND GRF MOLECULES IN COMBINATION THERAPY

(75) Inventor: CHRISTIAN MARSOLAIS, VILLE MONT-ROYAL (CA)

Correspondence Address: FISH & RICHARDSON P.C., P.O. BOX 1022, MINNEAPOLIS, MN, 55440-1022, US

(73) Assignee: THERATECHONOLGIES INC., MONTREAL (CA)

(21) Appl. No.: 12/763,978

(22) Filed: Apr. 20, 2010

Related U.S. Application Data
(60) Provisional application No. 61/170,862, filed on Apr. 20, 2009.

(51) Int. Cl. A61K 38/27; A61P 31/18; A61P 3/06

(52) U.S. Cl. 514/12

(57) ABSTRACT

Combination therapies comprising a protease inhibitor and a growth hormone (GH)-inducing compound (such as a GRF molecule) are described, in which there are no or substantially no drug interactions.

### CROSS REFERENCE TO RELATED APPLICATIONS

This application claims the benefit of U.S. Provisional Application Ser. No. 61/170,862, filed Apr. 20, 2009, which is herein incorporated by reference in its entirety.

### REFERENCE TO SEQUENCE LISTING

Pursuant to 37 C.F.R. 1.821(c), a sequence listing is submitted herewith as an ASCII compliant text file named “Sequence_listing.txt” which was created on Apr. 19, 2010 and has a size of 5725 bytes. The content of the aforementioned file named “Sequence_listing.txt” is hereby incorporated by reference in its entirety.

### FIELD OF THE INVENTION

The present invention relates to combination therapy for treatment of conditions, such as HIV infection and associated conditions, for example HIV-associated lipodystrophy. More specifically, the present invention is concerned with combination therapy comprising a plurality of compounds, at least one of which is a protease inhibitor and at least another of which is a growth hormone (GH)-inducing compound such as a GRF molecule.

### BACKGROUND OF THE INVENTION

Drug interactions are often observed in cases where multiple drugs are administered to a patient in a combination therapy. Such interactions may alter the pharmacokinetics and clearance of one or more of the drugs being administered, and as such the intended dosage and expected efficacy of a drug may be altered, and in some cases contraindications may occur resulting in potentially serious or life-threatening adverse effects.

Acquired immune deficiency syndrome (AIDS) is a disease of the human immune system caused by human immunodeficiency virus (HIV) infection. At the end of 2007, it was estimated that 33.2 million people were HIV positive, and during that year another 2.5 million people became infected with HIV and 2.1 million died of AIDS (according to estimates of WHO and UNAIDS).

Currently, HIV infection is treated with antiretroviral therapy (ART), which includes the use of protease inhibitors which inhibit HIV proteases. However, while a number of protease inhibitors have been developed for HIV treatment, they are known to suffer from drug interactions with other agents and may therefore be problematic when administered in parallel with other drug(s) for the treatment of other conditions or symptoms in HIV-infected patients, or for example in the context of a treatment regimen of a drug “cocktail” which is often of interest in HIV treatment. For example, various drug interactions have been reported with protease inhibitors such as ritonavir, at least partly due to its inhibitory effect on CYP3A4 (Zhou at al., Therapeutics and Clinical Risk Management 2005: 1(1): 3-13), which has therefore limited their use in conjunction with certain drugs. Table I provides examples of compounds, such as drugs/class of drugs, known to be associated with drug interactions with ritonavir.

TABLE I
Compounds/drugs known to be associated with drug interactions with ritonavir (from the Merck
Manuals Online Medical Library, http://www.merck.com/mmpe/lexicomp/ritonavir.html).

| | Risk | | Risk |
|---|---|---|---|
| | C = Monitor therapy | | C = Monitor therapy |
| | D = Consider therapy | | D = Consider therapy |
| | modification | | modification |
| Compounds/Drugs | X = Avoid combination | Drugs | E = Avoid combination |
| Abacavir | C | Metronidazole | C |
| Alfuzosin | X | Nebivolol | C |
| Almotriptan | D | Nefazodone | C |
| Alosetron | C | Nevirapine | D |
| Amiodarone | X | Nilotinib | X |
| Antacids | C | Nisoldipine | X |
| Antifungal Agents | D | Oral Contraceptive | D |
| (Azole Derivatives, | | (Estrogens) | |
| Systemic) | | | |
| Atomoxetine | D | Paricalcitol | C |
| Atovaquone | C | Pazopanib | D |
| Benzodiazepines | D | Peginterferon Alfa-2b | C |
| Bosentan | D | P-Glycoprotein Inducers | C |
| Brinzolamide | C | P-Glycoprotein | C |
| | | Inhibitors | |
| Bupropion | C | P-Glycoprotein | C |
| | | Substrates | |
| Calcium Channel | D | Phenytoin | D |
| Blockers | | | |
| (Dihydropyridine) | | | |
| Calcium Channel | D | Pimecrolimus | C |
| Blockers | | | |
| (Nondihydropyridine) | | | |
| Carbamazepine | D | Pimozide | X |
| Ciclesonide | C | Pitavastatin | X |
| Cisapride | X | Prasugrel | C |
| Clarithromycin | D | Propafenone | X |
| Codeine | D | Protease Inhibitors | D |
| Colchicine | D | Quinidine | X |
| Corticosteroids (Orally | D | Ranolazine | X |
| Inhaled) | | | |
| Cyclosporine | D | Rifamycin Derivatives | D |
| Cyclosporine, Systemic | D | Rivaroxaban | X |
| CYP2C8 Substrates | D | Romidepsin | X |
| (High risk) | | | |
| CYP2D6 Substrates | D | Salmeterol | X |
| CYP3A4 Inducers | C | Saxagliptin | D |
| (Strong) | | | |
| CYP3A4 Substrates | D | Sildenafil | D |
| Dabigatran Etexilate | X | Silodosin | X |
| Deferasirox | D | Sirolimus | C |
| Delavirdine | D | Sorafenib | C |
| Digoxin | C | St Johns Wort | X |
| Disulfiram | X | Tacrolimus | D |
| Divalproex | C | Tacrolimus, Systemic | D |
| Dronabinol | C | Tacrolimus, Topical | C |
| Dronedarone | X | Tadalafil | D |
| Dutasteride | C | Tamoxifen | X |
| Efavirenz | D | Tamsulosin | X |
| Enfuvirtide | C | Temsirolimus | D |
| Eplerenone | X | Tenofovir | C |
| Ergot Derivatives | X | Tetrabenazine | D |
| Etravirine | X | Theophylline | C |
| | | Derivatives | |
| Everolimus | X | Thioridazine | X |
| Fentanyl | D | Tolvaptan | X |
| Fesoterodine | D | Topotecan | X |
| Flecainide | X | Tramadol | C |
| Fusidic Acid | D | Trazodone | D |
| Garlic | C | Treprostinil | C |
| Guanfacine | C | Tricyclic | C |
| | | Antidepressants | |
| Halofantrine | X | Valproic Acid | C |
| HMG-CoA Reductase | D | Vardenafil | D |
| Inhibitors | | | |
| Ixabepilone | D | Vinblastine | D |
| Lamotrigine | D | Vincristine | D |
| Maraviroc | D | Voriconazole | X |
| Meperidine | D | Warfarin | C |
| Methadone | C | Zidovudine | C |

An example of a significant condition observed in a large percentage of HIV patients is HIV-associated lipodystrophy, a metabolic disorder characterized by fat accumulation and/or peripheral fat loss. In particular, HIV-infected patients treated with ART commonly experience changes in fat distribution that include increased visceral and central fat accumulation, as well as loss of extremity and subcutaneous fat (especially in the facial fat pads, limbs and buttocks) in association with insulin resistance and dyslipidemia. Recent data suggest increased cardiovascular diseases and myocardial infarction rates in patients treated with prolonged ART. Therefore, although ART regimens are desirable for the treatment of HIV infection, they have the undesirable side effect of aggravating fat accumulation and/or loss in patients.

There is therefore a need to identify therapeutic strategies which may be used for treatment of various conditions suffered by HIV-infected patients, in which multiple drugs may be used to treat such conditions with minimal drug interactions. For example, it is desirable to identify therapeutic approaches to treat such conditions, which are compatible with protease inhibitor treatment and without incurring any significant negative effects or loss of efficacy.

The present description refers to a number of documents, the content of which is herein incorporated by reference in their entirety.

### SUMMARY OF THE INVENTION

The present invention relates to combination therapy for treatment of a condition, such as HIV infection and associated conditions, including HIV-associated lipodystrophy. More specifically, the present invention is concerned with combination therapy comprising a plurality of compounds, at least one of which is a protease inhibitor and at least another of which is a growth hormone (GH)-inducing compound such as a GRF molecule.

More specifically, in accordance with the present invention, there is provided a method of inducing GH levels in a subject undergoing an antiretroviral protease inhibitor treatment regimen or who is a candidate for an antiretroviral protease inhibitor treatment regimen, without significantly affecting pharmacokinetics or clearance of said protease inhibitor, said method comprising administering to said subject an effective amount of (hexenoyl trans-3)hGRF(1-44)NH₂.

The invention further provides a method of inducing GH levels in a subject undergoing an antiretroviral protease inhibitor treatment regimen or who is a candidate for an antiretroviral protease inhibitor treatment regimen, without modifying said treatment regimen, said method comprising administering to said subject an effective amount of (hexenoyl trans-3)hGRF(1-44)NH₂.

The invention further provides a method of providing GH therapy to a subject undergoing antiretroviral protease inhibitor treatment regimen or who is a candidate for antiretroviral protease inhibitor treatment regimen, without significantly affecting pharmacokinetics or clearance of said inhibitor, said method comprising administering to said subject an effective amount of (hexenoyl trans-3)hGRF(1-44)NH₂.

The invention further provides a method of providing GH therapy to a subject undergoing an antiretroviral protease inhibitor treatment regimen or who is a candidate for an antiretroviral protease inhibitor treatment regiment, without modifying said treatment regimen, said method comprising administering to said subject an effective amount of (hexenoyl trans-3)hGRF(1-44)NH₂.

The invention further provides a method of treating a condition associated with fat accumulation or hypercholesterolemia in a subject undergoing an antiretroviral protease inhibitor treatment regimen or who is a candidate for an antiretroviral protease inhibitor treatment regimen, without significantly affecting pharmacokinetics or clearance of said protease inhibitor, said method comprising administering to said subject an effective amount of (hexenoyl trans-3)hGRF(1-44)NH₂.

The invention further provides a method of treating a condition associated with fat accumulation or hypercholesterolemia in a subject undergoing an antiretroviral protease inhibitor treatment regimen or who is a candidate for an antiretroviral protease inhibitor treatment regiment, without modifying said treatment regimen, said method comprising administering to said subject an effective amount of (hexenoyl trans-3)hGRF(1-44)NH₂.

The invention further provides a method of treating excess abdominal fat in a HIV-infected subject with lipodystrophy in a subject undergoing antiretroviral protease inhibitor treatment regimen or who is a candidate for antiretroviral protease inhibitor treatment regimen, without significantly affecting pharmacokinetics or clearance of said inhibitor, said method comprising administering to said subject an effective amount of (hexenoyl trans-3)hGRF(1-44)NH₂.

The invention further provides a method of treating excess abdominal fat in a HIV-infected subject with lipodystrophy, wherein said subject is undergoing an antiretroviral protease inhibitor treatment regimen or is a candidate for an antiretroviral protease inhibitor treatment regimen, without substantially modifying said treatment regimen, said method comprising administering to said subject an effective amount of (hexenoyl trans-3)hGRF(1-44)NH₂.

The invention further provides a method comprising providing information to a subject or to a caregiver of the subject that (hexenoyl trans-3)hGRF(1-44)NH₂ and a protease inhibitor can be co-administered to the subject.

The invention further provides a package comprising: (a) (hexenoyl trans-3)hGRF(1-44)NH₂; and (b) information that (hexenoyl trans-3)hGRF(1-44)NH₂ and a protease inhibitor can be co-administered to a subject.

The invention further provides a use of (hexenoyl trans-3)hGRF(1-44)NH₂ for inducing GH levels in a subject undergoing antiretroviral protease inhibitor treatment regimen or who is a candidate for antiretroviral protease inhibitor treatment regimen, wherein said use does not significantly affect pharmacokinetics or clearance of said protease inhibitor.

The invention further provides a use of (hexenoyl trans-3)hGRF(1-44)NH₂ for the manufacture of a medicament for inducing GH levels in a subject undergoing antiretroviral protease inhibitor treatment regimen or who is a candidate for antiretroviral protease inhibitor treatment regimen, wherein said use does not significantly affect pharmacokinetics or clearance of said protease inhibitor.

The invention further provides a use of (hexenoyl trans-3)hGRF(1-44)NH₂ for inducing GH levels in a subject undergoing antiretroviral protease inhibitor treatment regimen or who is a candidate for antiretroviral protease inhibitor treatment regimen, without modifying said treatment regimen.

The invention further provides a use of (hexenoyl trans-3)hGRF(1-44)NH₂ for the manufacture of a medicament for inducing GH levels in a subject undergoing antiretroviral protease inhibitor treatment regimen or who is a candidate for antiretroviral protease inhibitor treatment regimen, without modifying said treatment regimen.

The invention further provides a use of (hexenoyl trans-3)hGRF(1-44)NH₂ for providing GH therapy to a subject undergoing antiretroviral protease inhibitor treatment regimen or who is a candidate for antiretroviral protease inhibitor treatment regimen, wherein said use does not significantly affect pharmacokinetics or clearance of said protease inhibitor.

The invention further provides a use of (hexenoyl trans-3)hGRF(1-44)NH₂ for the manufacture of a medicament for providing GH therapy to a subject undergoing antiretroviral protease inhibitor treatment regimen or who is a candidate for antiretroviral protease inhibitor treatment regimen, wherein said use does not significantly affect pharmacokinetics or clearance of said protease inhibitor.

The invention further provides a use of (hexenoyl trans-3)hGRF(1-44)NH₂ for providing GH therapy to a subject undergoing antiretroviral protease inhibitor treatment regimen or who is a candidate for antiretroviral protease inhibitor treatment regimen, without modifying said treatment regimen.

The invention further provides a use of (hexenoyl trans-3)hGRF(1-44)NH₂ for the manufacture of a medicament for providing GH therapy to a subject undergoing antiretroviral protease inhibitor treatment regimen or who is a candidate for antiretroviral protease inhibitor treatment regimen, without modifying said treatment regimen.

The invention further provides a use of (hexenoyl trans-3)hGRF(1-44)NH₂ for treating a condition associated with fat accumulation or hypercholesterolemia in a subject undergoing antiretroviral protease inhibitor treatment regimen or who is a candidate for antiretroviral protease inhibitor treatment regimen, wherein said use does not significantly affect pharmacokinetics or clearance of said protease inhibitor.

The invention further provides a use of (hexenoyl trans-3)hGRF(1-44)NH₂ for the manufacture of a medicament for treating a condition associated with fat accumulation or hypercholesterolemia in a subject undergoing antiretroviral protease inhibitor treatment regimen or who is a candidate for antiretroviral protease inhibitor treatment regimen, wherein said use does not significantly affect pharmacokinetics or clearance of said protease inhibitor.

The invention further provides a use of (hexenoyl trans-3)hGRF(1-44)NH₂ for treating a condition associated with fat accumulation or hypercholesterolemia in a subject undergoing antiretroviral protease inhibitor treatment regimen or who is a candidate for antiretroviral protease inhibitor treatment regimen, without modifying said treatment regimen.

The invention further provides a use of (hexenoyl trans-3)hGRF(1-44)NH₂ for the manufacture of a medicament for treating a condition associated with fat accumulation or hypercholesterolemia in a subject undergoing antiretroviral protease inhibitor treatment regimen or who is a candidate for antiretroviral protease inhibitor treatment regimen, without modifying said treatment regimen.

The invention further provides a use of (hexenoyl trans-3)hGRF(1-44)NH₂ for treating excess abdominal fat in a HIV-infected subject with lipodystrophy, wherein said subject is undergoing an antiretroviral protease inhibitor treatment regimen or is a candidate for antiretroviral protease inhibitor treatment regimen, wherein said use does not significantly affect pharmacokinetics or clearance of said protease inhibitor.

The invention further provides a use of (hexenoyl trans-3)hGRF(1-44)NH₂ for the manufacture of a medicament for treating excess abdominal fat in a HIV-infected subject with lipodystrophy, wherein said subject is undergoing an antiretroviral protease inhibitor treatment regimen or is a candidate for antiretroviral protease inhibitor treatment regimen, wherein said use does not significantly affect pharmacokinetics or clearance of said protease inhibitor.

The invention further provides a use of (hexenoyl trans-3)hGRF(1-44)NH₂ for treating excess abdominal fat in a HIV-infected subject with lipodystrophy, wherein said subject is undergoing an antiretroviral protease inhibitor treatment regimen or is a candidate for antiretroviral protease inhibitor treatment regimen, without modifying said treatment regimen.

The invention further provides a use of (hexenoyl trans-3)hGRF(1-44)NH₂ for the manufacture of a medicament for treating excess abdominal fat in a HIV-infected subject with lipodystrophy, wherein said subject is undergoing an antiretroviral protease inhibitor treatment regimen or is a candidate for antiretroviral protease inhibitor treatment regimen, without modifying said treatment regimen.

The invention further provides (hexenoyl trans-3)hGRF(1-44)NH₂ for inducing GH levels in a subject undergoing antiretroviral protease inhibitor treatment regimen or who is a candidate for antiretroviral protease inhibitor treatment regimen, wherein said (hexenoyl trans-3)hGRF(1-44)NH₂ does not significantly affect pharmacokinetics or clearance of said protease inhibitor.

The invention further provides (hexenoyl trans-3)hGRF(1-44)NH₂ for the manufacture of a medicament for inducing GH levels in a subject undergoing antiretroviral protease inhibitor treatment regimen or who is a candidate for antiretroviral protease inhibitor treatment regimen, wherein said (hexenoyl trans-3)hGRF(1-44)NH₂ does not significantly affect pharmacokinetics or clearance of said protease inhibitor.

The invention further provides (hexenoyl trans-3)hGRF(1-44)NH₂ for inducing GH levels in a subject undergoing antiretroviral protease inhibitor treatment regimen or who is a candidate for antiretroviral protease inhibitor treatment regimen, without modifying said treatment regimen.

The invention further provides (hexenoyl trans-3)hGRF(1-44)NH₂ for the manufacture of a medicament for inducing GH levels in a subject undergoing antiretroviral protease inhibitor treatment regimen or who is a candidate for antiretroviral protease inhibitor treatment regimen, without modifying said treatment regimen.

The invention further provides (hexenoyl trans-3)hGRF(1-44)NH₂ for providing GH therapy to a subject undergoing antiretroviral protease inhibitor treatment regimen or who is a candidate for antiretroviral protease inhibitor treatment regimen, wherein said (hexenoyl trans-3)hGRF(1-44)NH₂ does not significantly affect pharmacokinetics or clearance of said protease inhibitor.

The invention further provides (hexenoyl trans-3)hGRF(1-44)NH₂ for the manufacture of a medicament for providing GH therapy to a subject undergoing antiretroviral protease inhibitor treatment regimen or who is a candidate for antiretroviral protease inhibitor treatment regimen, wherein said (hexenoyl trans-3)hGRF(1-44)NH₂ does not significantly affect pharmacokinetics or clearance of said protease inhibitor.

The invention further provides (hexenoyl trans-3)hGRF(1-44)NH₂ for providing GH therapy to a subject undergoing antiretroviral protease inhibitor treatment regimen or who is a candidate for antiretroviral protease inhibitor treatment regimen, without modifying said treatment regimen.

The invention further provides (hexenoyl trans-3)hGRF(1-44)NH₂ for the manufacture of a medicament for providing GH therapy to a subject undergoing antiretroviral protease inhibitor treatment regimen or who is a candidate for antiretroviral protease inhibitor treatment regimen, without modifying said treatment regimen.

The invention further provides (hexenoyl trans-3)hGRF(1-44)NH₂ for treating a condition associated with fat accumulation or hypercholesterolemia in a subject undergoing antiretroviral protease inhibitor treatment regimen or who is a candidate for antiretroviral protease inhibitor treatment regimen, wherein said (hexenoyl trans-3)hGRF(1-44)NH₂ does not significantly affect pharmacokinetics or clearance of said protease inhibitor.

The invention further provides (hexenoyl trans-3)hGRF(1-44)NH₂ for the manufacture of a medicament for treating a condition associated with fat accumulation or hypercholesterolemia in a subject undergoing antiretroviral protease inhibitor treatment regimen or who is a candidate for antiretroviral protease inhibitor treatment regimen, wherein said (hexenoyl trans-3)hGRF(1-44)NH₂ does not significantly affect pharmacokinetics or clearance of said protease inhibitor.

The invention further provides (hexenoyl trans-3)hGRF(1-44)NH₂ for treating a condition associated with fat accumulation or hypercholesterolemia in a subject undergoing antiretroviral protease inhibitor treatment regimen or who is a candidate for antiretroviral protease inhibitor treatment regimen, without modifying said treatment regimen.

The invention further provides (hexenoyl trans-3)hGRF(1-44)NH₂ for the manufacture of a medicament for treating a condition associated with fat accumulation or hypercholesterolemia in a subject undergoing antiretroviral protease inhibitor treatment regimen or who is a candidate for antiretroviral protease inhibitor treatment regimen, without modifying said treatment regimen.

The invention further provides (hexenoyl trans-3)hGRF(1-44)NH₂ for treating excess abdominal fat in a HIV-infected subject with lipodystrophy, wherein said subject is undergoing an antiretroviral protease inhibitor treatment regimen or is a candidate for antiretroviral protease inhibitor treatment regimen, wherein said (hexenoyl trans-3)hGRF(1-44)NH₂ does not significantly affect pharmacokinetics or clearance of said protease inhibitor.

The invention further provides (hexenoyl trans-3)hGRF(1-44)NH₂ for the manufacture of a medicament for treating excess abdominal fat in a HIV-infected subject with lipodystrophy, wherein said subject is undergoing an antiretroviral protease inhibitor treatment regimen or is a candidate for antiretroviral protease inhibitor treatment regimen, wherein said (hexenoyl trans-3)hGRF(1-44)NH₂ does not significantly affect pharmacokinetics or clearance of said protease inhibitor.

The invention further provides (hexenoyl trans-3)hGRF(1-44)NH₂ for treating excess abdominal fat in a HIV-infected subject with lipodystrophy, wherein said subject is undergoing an antiretroviral protease inhibitor treatment regimen or is a candidate for antiretroviral protease inhibitor treatment regimen, without modifying said treatment regimen.

The invention further provides (hexenoyl trans-3)hGRF(1-44)NH₂ for the manufacture of a medicament for treating excess abdominal fat in a HIV-infected subject with lipodystrophy, wherein said subject is undergoing an antiretroviral protease inhibitor treatment regimen or is a candidate for antiretroviral protease inhibitor treatment regimen, without modifying said treatment regimen.

The invention further provides a composition comprising (hexenoyl trans-3)hGRF(1-44)NH₂ and a pharmaceutically acceptable excipient for inducing GH levels in a subject undergoing antiretroviral protease inhibitor treatment regimen or who is a candidate for antiretroviral protease inhibitor treatment regimen, wherein said (hexenoyl trans-3)hGRF(1-44)NH₂ does not significantly affect pharmacokinetics or clearance of said protease inhibitor.

The invention further provides a composition comprising (hexenoyl trans-3)hGRF(1-44)NH₂ and a pharmaceutically acceptable excipient for inducing GH levels in a subject undergoing antiretroviral protease inhibitor treatment regimen or who is a candidate for antiretroviral protease inhibitor treatment regimen, without modifying said treatment regimen.

The invention further provides a composition comprising (hexenoyl trans-3)hGRF(1-44)NH₂ and a pharmaceutically acceptable excipient for providing GH therapy to a subject undergoing antiretroviral protease inhibitor treatment regimen or who is a candidate for antiretroviral protease inhibitor treatment regimen, wherein said (hexenoyl trans-3)hGRF(1-44)NH₂ does not significantly affect pharmacokinetics or clearance of said protease inhibitor.

The invention further provides a composition comprising (hexenoyl trans-3)hGRF(1-44)NH₂ and a pharmaceutically acceptable excipient for providing GH therapy to a subject undergoing antiretroviral protease inhibitor treatment regimen or who is a candidate for antiretroviral protease inhibitor treatment regimen, without modifying said treatment regimen.

The invention further provides a composition comprising (hexenoyl trans-3)hGRF(1-44)NH₂ and a pharmaceutically acceptable excipient for treating a condition associated with fat accumulation or hypercholesterolemia in a subject undergoing antiretroviral protease inhibitor treatment regimen or who is a candidate for antiretroviral protease inhibitor treatment regimen, wherein said (hexenoyl trans-3)hGRF(1-44)NH₂ does not significantly affect pharmacokinetics or clearance of said protease inhibitor.

The invention further provides a composition comprising (hexenoyl trans-3)hGRF(1-44)NH₂ and a pharmaceutically acceptable excipient for treating a condition associated with fat accumulation or hypercholesterolemia in a subject undergoing antiretroviral protease inhibitor treatment regimen or who is a candidate for antiretroviral protease inhibitor treatment regimen, without modifying said treatment regimen.

The invention further provides a composition comprising (hexenoyl trans-3)hGRF(1-44)NH₂ and a pharmaceutically acceptable excipient for treating HIV-associated lipodystrophy in a subject undergoing antiretroviral protease inhibitor treatment regimen or who is a candidate for antiretroviral protease inhibitor treatment regimen, wherein said (hexenoyl trans-3)hGRF(1-44)NH₂ does not significantly affect pharmacokinetics or clearance of said protease inhibitor.

The invention further provides a composition comprising (hexenoyl trans-3)hGRF(1-44)NH₂ and a pharmaceutically acceptable excipient for treating HIV-associated lipodystrophy in a subject undergoing antiretroviral protease inhibitor treatment regimen or who is a candidate for antiretroviral protease inhibitor treatment regimen, without modifying said treatment regimen.

In an embodiment, the above-mentioned method, use, (hexenoyl trans-3)hGRF(1-44)NH₂ or composition further comprises, prior to said treating, selecting a subject who is undergoing or who is a candidate for an antiretroviral protease inhibitor treatment regimen.

In an embodiment, the above-mentioned method further comprises providing information to the subject or to the caregiver of the subject that (hexenoyl trans-3)hGRF(1-44)NH₂ can be co-administered to the subject without affecting pharmacokinetics of said protease inhibitor. In another embodiment, the above-mentioned method further comprises providing information to the subject or to the caregiver of the subject that (hexenoyl trans-3)hGRF(1-44)NH₂ can be co-administered to the subject without modifying the treatment regimen of said protease inhibitor.

In an embodiment, the above-mentioned protease inhibitor is ritonavir.

In another embodiment, the above-mentioned (hexenoyl trans-3)hGRF(1-44)NH₂ is administered, or is adapted for administration, at a daily dose of about 2 mg.

In another embodiment, the above-mentioned (hexenoyl trans-3)hGRF(1-44)NH₂ is administered subcutaneously, or is adapted for subcutaneous administration.

In an embodiment, the above-mentioned package further comprises information that no modification of the treatment regiment of the protease inhibitor is necessary during the co-administration.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows a concentration-time profile of ritonavir with (Treatment A) and without (Treatment B) pre-treatment with (hexenoyl trans-3)hGRF(1-44)NH₂.

### DESCRIPTION OF ILLUSTRATIVE EMBODIMENTS

The present invention relates to combination therapies utilizing a protease inhibitor. In an embodiment, the present invention relates to a combined therapy comprising a protease inhibitor (e.g., ritonavir) and a growth hormone releasing factor (GRF) or GRF analog (e.g., (hexenoyl trans-3)hGRF(1-44)NH₂). In embodiments, such therapies relate to the treatment of a condition associated with fat accumulation and/or redistribution, such as HIV-associated lipodystrophy.

In embodiments, the invention relates to a combination therapy comprising administering an effective amount of GRF or an analog thereof to a subject who is undergoing treatment with a protease inhibitor or who is a candidate for treatment with a protease inhibitor.

Growth hormone (GH) or somatotropin is secreted by the pituitary gland. Its activity is fundamental for the linear growth of a young organism but also for the maintenance of the integrity at its adult state. GH acts directly or indirectly on the peripheral organs by stimulating the synthesis of growth factors (insulin-like growth factor-1 or IGF-I) or of their receptors (epidermal growth factor or EGF). The direct action of GH is of the type referred to as anti-insulinic, which favors the lipolysis at the level of adipose tissues. Through its action on IGF-I (somatomedin C) synthesis and secretion, GH stimulates the growth of cartilage and the bones (structural growth), protein synthesis and cellular proliferation in multiple peripheral organs, including muscle and skin. In adults, GH participates in the maintenance of a protein anabolism state and plays a primary role in the tissue regeneration phenomenon after a trauma.

The secretion of GH by the pituitary gland is principally controlled by two hypothalamic peptides, somatostatin and growth hormone-releasing hormone (GHRH; also known as growth hormone-releasing factor or GRF). Somatostatin inhibits its secretion, whereas GRF stimulates it.

Among all known GRF molecules, GRF analogs containing a hydrophobic tail as defined in the present application include modified versions or analogs of human GRF that have been shown to have higher proteolytic stability in biological milieu and as a result, these analogs were shown to display longer duration of action resulting in enhanced growth hormone secretion and insulin like growth factor-1 synthesis (U.S. Pat. Nos. 5,861,379 and 5,939,386). Due to their superior plasma stability and pharmacological properties compared to the native GRF (1-44) amide, these GRF analogs were shown to confer therapeutic efficacy in several medical conditions, e.g., wasting associated with COPD (International Application No. WO 05/037307), recovery after hip fracture, frailty in elderly population, enhancing immune response and HIV-associated lipodystrophy (U.S. Pat. No. 7,316,997).

The term “GRF molecule” as used in the context of the present invention includes, without limitation, human native GRF (amino acids 1-44, SEQ ID NO: 3) and fragments (1-40), (1-29 [SEQ ID NO: 5]), fragments ranging between 1-29 and the 1-44 sequence, and any other fragments; GRF from other species and fragments thereof; GRF variants containing amino acid(s) substitution(s), addition(s) and/or deletion(s) such that the amino acid sequence of the variant has at least about 90% of homology with the native amino acid sequence, in an embodiment at least about 95% of homology with the native amino acid sequence. In an embodiment, the above-mentioned fragments/variants retain at least about 10% of the activity of stimulating GH secretion as compared to the native GRF; derivatives or analogs of GRF or fragments or variants thereof having for a example an organic group or a moiety coupled to the GRF amino acid sequence at the N-terminus, the C-terminus or on the side-chain (e.g., human native GRF having a C-terminal unsubstituted amide moiety, SEQ ID NO: 2; fragment 1-29 of human native GRF having a C-terminal unsubstituted amide moiety, SEQ ID NO: 4); and salts of GRF (human or from other species), as well as salts of GRF fragments, variants, analogs and derivatives. The GRF molecules of the present invention also encompass the GRF molecules currently known in the art, including, without limitation, the albumin-conjugated GRF (U.S. Pat. No. 7,268,113); pegylated GRF peptide (U.S. Pat. Nos. 7,256,258 and 6,528,485); porcine GRF (1-40) (U.S. Pat. No. 6,551,996); canine GRF (U.S. patent application no. 2005/0064554); GRF variants of 1-29 to 1-44 amino acid length (U.S. Pat. Nos. 5,846,936, 5,696,089, 5,756,458 and 5,416,073, and U.S. patent application Nos. 2006/0128615 and 2004/0192593); and Pro⁰-GRF peptide and variants thereof (U.S. Pat. No. 5,137,872).

The GRF analogs include those described in U.S. Pat. Nos. 5,681,379 and 5,939,386, which also describe their method of synthesis. More particularly, these GRF analogs are defined by the following formula A:

X-GRF Peptide(A)

The GRF peptide is a peptide of the following formula B:

(SEQ ID NO: 1)

A1-A2-Asp-Ala-Ile-Phe-Thr-A8-Ser-Tyr-Arg-Lys-A13-
Leu-A15-Gln-Leu-A18-Ala-Arg-Lys-Leu-Leu-A24-A25-
Ile-A27-A28-Arg-A30-R0 (B)

wherein,
A1 is Tyr or His;
A2 is Val or Ala;
A8 is Asn or Ser;
A13 is Val or Ile;
A15 is Ala or Gly;
A18 is Ser or Tyr;
A24 is Gln or His;
A25 is Asp or Glu;
A27 is Met, Ile or Nle
A28 is Ser or Asn;
A30 is a bond or amino acid sequence of 1 up to 15 residues; and
R0 is NH₂ or NH—(CH₂)n-CONH₂, with n=1 to 12.
   wherein X is:
   (a) a hydrophobic tail anchored via an amide bond to the N-terminus of the peptide, said hydrophobic tail comprising (i) a backbone of 5 to 7 atoms;
   wherein said backbone can be substituted by C₁₋₆ alkyl, C₃₋₆ cycloalkyl, or C₆₋₁₂ aryl, and (ii) at least one rigidifying moiety connected to at least two atoms of the backbone; the rigidifying moiety being a double bond, a triple bond, a saturated or unsaturated C₃₋₉ cycloalkyl, or a C₆₋₁₂ aryl; or
   (b) a moiety selected from:

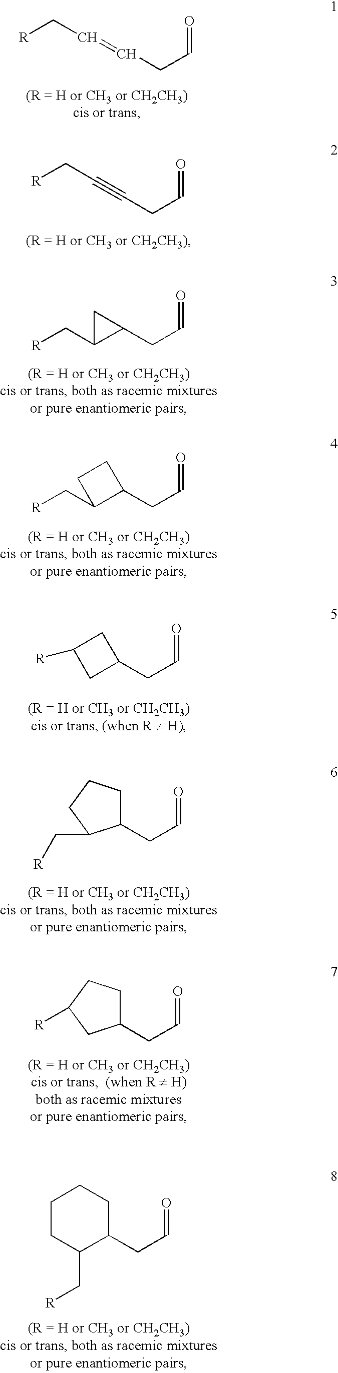

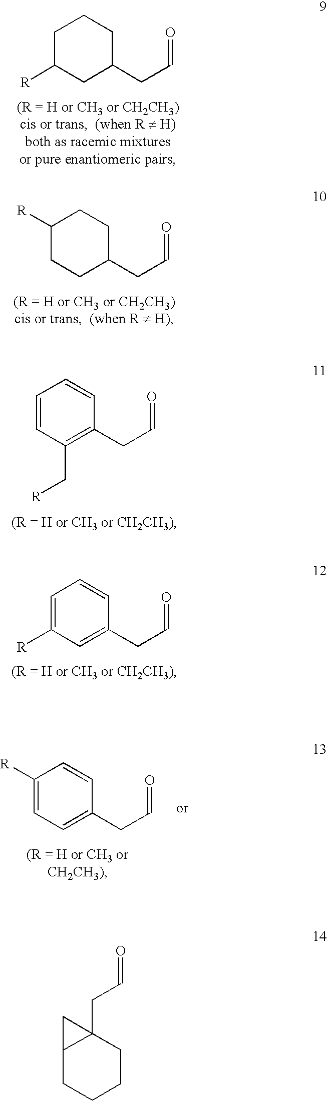

In an embodiment, group X is:

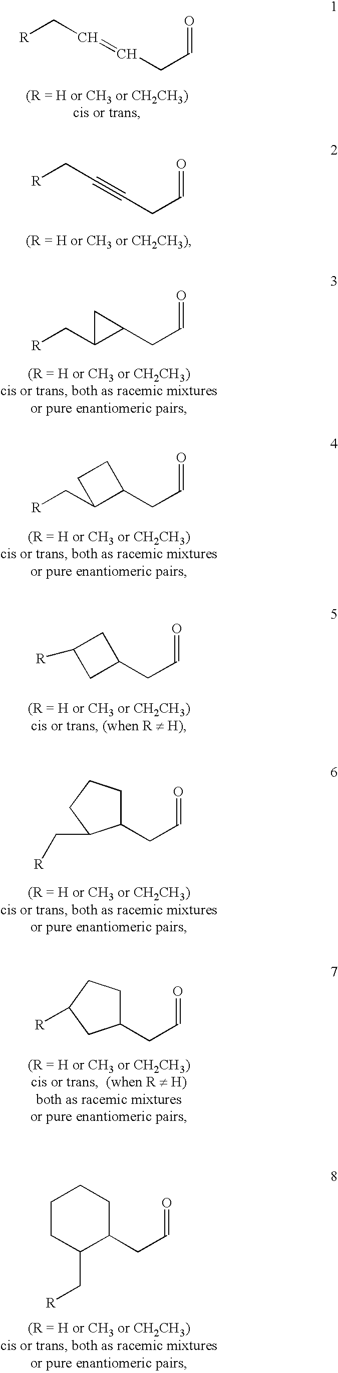

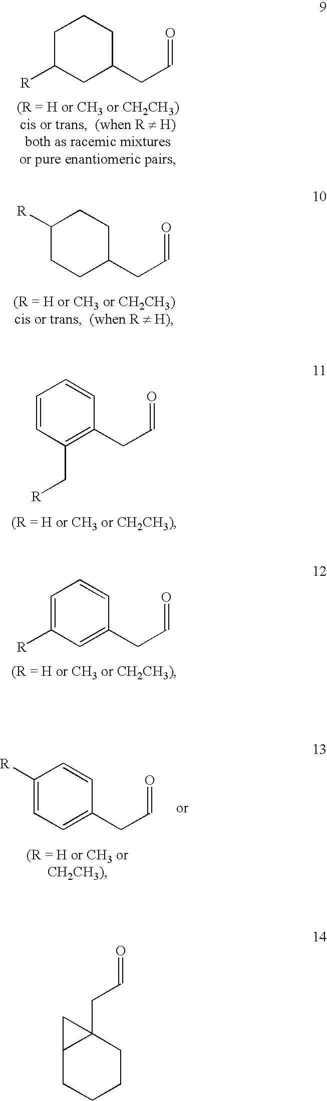

In an embodiment, in formula B, A30 is:
(a) a bond,
(b) an amino acid sequence corresponding to positions 30-44 of a natural GRF peptide (e.g., positions 30-44 of human GRF peptide, SEQ ID NO: 6), or
(c) the amino acid sequence of (b) having a 1-14 amino acid deletion from its C-terminus.

In an embodiment, the GRF molecule is (hexenoyl trans-3)hGRF(1-44)NH₂ (also referred to as trans-3-hexenoyl]hGRF (1-44) amide or TH9507 herein). (hexenoyl trans-3)hGRF(1-44)NH₂ is a synthetic human growth hormone releasing factor analog that comprises the 44-amino acid sequence of human growth hormone releasing factor (hGRF) on which a hexenoyl moiety, a C₆ side chain, has been anchored on Tyr1 at the N-terminus.

(trans-3-hexenoyl)hGRF (1-44)NH₂ has the following structure (SEQ ID NO: 7):

(trans)CH₃—CH₂—CH═CH—CH₂—CO-Tyr-Ala-Asp-Ala-Ile-
Phe-Thr-Asn-Ser-Tyr-Arg-Lys-Val-Leu-Gly-Gln-Leu-
Ser-Ala-Arg-Lys-Leu-Leu-Gln-Asp-Ile-Met-Ser-Arg-
Gln-Gln-Gly-Glu-Ser-Asn-Gln-Glu-Arg-Gly-Ala-Arg-
Ala-Arg-Leu-NH₂.

“GH therapy” as used herein refers to treatment which results in an increase in GH levels in a subject. In an embodiment, the subject may exhibit a GH deficiency (i.e., lower than normal levels of GH) and therefore such GH therapy is effected to increase GH levels with a view to reverse such deficiency. In a further embodiment, the subject may exhibit normal GH levels and therefore such GH therapy is effected to increase GH levels to result in higher than normal GH levels. GH therapy may in embodiments be achieved by administration of GH or a fragment, variant or analog thereof, and in further embodiments may be achieved by administering a compound which induces GH secretion, e.g., a GH secretagogue, GRF, or a GRF molecule. “Compound which induces GH secretion” as used herein refers to any compound or molecule, natural or synthetic, which may result in, either directly or indirectly, GH secretion and/or an increase in GH secretion. In an embodiment, the compound which induces GH secretion is a GRF molecule.

In embodiments, a GRF molecule may be used to treat a condition such as HIV-associated lipodystrophy, HIV-lipohypertrophy, abdominal obesity, GH deficiency, frailty, mild cognitive impairment, immune deficiency, wasting associated with a chronic condition or long-term condition, or malnutrition associated with a chronic condition or a long-term condition. Chronic conditions include, without limitation, HIV infection, AIDS, cystic fibrosis, chronic obstructive pulmonary disease (COPD), hip fracture, trauma, and major surgery.

In further embodiments, a GRF molecule may be used to treat a condition associated with fat accumulation. Fat accumulation is observed in a range of conditions or syndromes such as obesity, metabolic syndrome (also known as syndrome X), and excess abdominal fat in a HIV-infected patient with lipodystrophy (HIV-associated lipodystrophie). All these conditions include features which are known to increase the risk of diabetes and/or cardiovascular diseases.

“Protease inhibitor” as used herein refers to any compound which may be used to directly or indirectly inhibit protease activity necessary for normal retroviral function/replication/infection/propagation. For example, protease inhibitors include those which directly inhibit HIV proteases, as well as those which indirectly inhibit HIV proteases. Such indirect inhibition of an HIV protease may be for example via inhibition of other enzyme(s) that metabolize other protease inhibitors which inhibit HIV proteases, thus increasing the level of and potentiating the effect of such other protease inhibitors. Various protease inhibitors are known in the art and are used in treatment regimens for patients suffering from HIV infection and AIDS (e.g., in the context of ART). Examples of protease inhibitors include Saquinavir (Fortovase™, Invirase™), Indinavir (Crixivan™), Ritonavir (Norvir™), Nelfinavir (Viracept™), Amprenavir (Agenerase™), Lopinavir (Kaletra™), Atazanavir (Reyataz™), Fosamprenavir (Lexiva™, Telzir™), Tipranavir (Aptivus™) and Darunavir (Prezista™). In an embodiment, the protease inhibitor is ritonavir (Norvir™, CAS number: 155213-67-5); IUPAC name: 1,3-thiazol-5-ylmethyl N-[(2S,3S,5S)-3-hydroxy-5-[(2S)-3-methyl-2-{[methyl({[2-(propan-2-yl)-1,3-thiazol-4-yl]methyl})carbamoyl]amino}butanamido]-1,6-diphenylhexan-2-yl]carbamate).

“Pharmacokinetics” as used herein refers to the parameters of absorption and distribution of an administered drug, such as the rate at which a drug action begins and the duration of the effect, the concentration of the drug in tissues, organs, body fluids (e.g., blood, plasma or urine concentration), the chemical modifications of the substance in the body (e.g., by liver enzymes, such as the CYP system) and the effects and routes of excretion of the metabolites of the drug. In an embodiment, the pharmacokinetics comprises the blood or plasma concentration.

The expression “without modifying said treatment regimen” means that the administration of the GRF molecule (e.g., (hexenoyl trans-3)hGRF(1-44)NH₂) to the subject does not require any modifications to the protease inhibitor-based treatment regimen, i.e. there is no need to:
stop or temporarily delay protease inhibitor treatment;
replace the protease inhibitor administered with another protease inhibitor or different drug;
change the dosage, or change the frequency of administration of the protease inhibitor; and/or
monitor possible drug interactions and/or changes in the efficacy of the treatment.

In embodiments, the invention provides a combination therapy comprising a use of (a) a GRF molecule and a protease inhibitor; (b) a composition comprising a GRF molecule and a pharmaceutically acceptable carrier and a composition comprising a protease inhibitor and a pharmaceutically acceptable carrier; or (c) a composition comprising a GRF molecule, a protease inhibitor, and in an embodiment further comprising a pharmaceutically acceptable carrier.

“Combination therapy” as used herein refers to administration of two or more compounds or compositions to a subject, for example a GRF molecule or a composition comprising a GRF molecule, and a protease inhibitor or a composition comprising a protease inhibitor. In embodiments, the combination therapy may be administered sequentially or simultaneously. For example, in an embodiment the GRF molecule or composition comprising the GRF molecule may be administered to a subject undergoing treatment with a protease inhibitor, i.e., to which a protease inhibitor has already been administered. In a further embodiment, the GRF molecule or composition comprising the GRF molecule may be administered to a subject who is a candidate for treatment with a protease inhibitor, i.e., a subject who has been identified as one who may benefit from protease inhibitor therapy, and thus to which a protease inhibitor may be administered at a later time. In a further embodiment the GRF molecule and protease inhibitor (or compositions thereof) may be administered at substantially the same time, either via separate administration or administered together in the same composition.

As noted above, in various embodiments, the above-mentioned GRF molecule and protease inhibitor may be used therapeutically in compositions, formulations or medicaments to effect the above-noted combination therapy or to prevent or treat the above-noted conditions. The invention provides corresponding methods of medical treatment, in which a therapeutic dose of a GRF molecule and/or a protease inhibitor is administered in a pharmacologically acceptable formulation(s), e.g. to a patient or subject in need thereof. Accordingly, the invention also provides therapeutic compositions comprising a GRF molecule and/or a protease inhibitor and a pharmacologically acceptable excipient or carrier. In an embodiment, such compositions include the GRF molecule and/or protease inhibitor in a therapeutically or prophylactically effective amount sufficient to effect the above-noted combined therapy and to prevent or treat the above-noted conditions. The composition may be soluble in an aqueous solution at a physiologically acceptable pH.

A “therapeutically effective amount” refers to an amount effective, at dosages and for periods of time necessary, to achieve the desired therapeutic result, such as to effect the above-noted combination therapy or to prevent or treat the above-noted conditions, in a subject in need thereof. A therapeutically effective amount of a GRF molecule or a protease inhibitor may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as preventing or inhibiting the rate of onset or progression of the above-noted conditions. A prophylactically effective amount can be determined as described above for the therapeutically effective amount. For any particular subject, specific dosage regimens may be adjusted over time according to the individual need and the professional judgement of the person administering or supervising the administration of the compositions.

As used herein “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, subcutaneous, sublingual or oral administration. Pharmaceutically acceptable carriers include sterile aqueous solutions or dispersions and sterile powders for the preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated.

Therapeutic compositions typically must be sterile and stable under the conditions of manufacture and storage. The composition can be formulated as a solution, microemulsion, liposome, or other ordered structure suitable to high drug concentration. Moreover, a GRF molecule or protease inhibitor can be administered in a time release formulation (e.g., sustained release, controlled release, delayed release). The active compounds can be prepared with carriers that will protect the compound against rapid release, such as a controlled release formulation. Many methods for the preparation of such formulations are generally known to those skilled in the art.

Sterile injectable solutions can be prepared by incorporating the active compound (e.g. a GRF molecule or protease inhibitor) in the required amount in an appropriate solvent with one or a combination of excipients, as required, followed by filtered sterilization. Generally, dispersions are prepared by incorporating the active compound into a sterile vehicle which contains a basic dispersion medium and other excipient(s). In the case of sterile powders for the preparation of sterile injectable solutions, the preferred methods of preparation are vacuum drying and freeze-drying (lyophilization) which yields a powder of the active ingredient plus any additional desired ingredient from a previously sterile-filtered solution thereof. In accordance with an alternative aspect of the invention, a GRF molecule or protease inhibitor may be formulated with one or more additional compounds that enhance its solubility.

In accordance with another aspect of the invention, therapeutic compositions of the present invention, comprising a GRF molecule and/or protease inhibitor, may be provided in containers, kits or packages (e.g., commercial packages) which further comprise instructions for its use for the above-noted combination therapy or to prevent or treat the above-noted conditions.

Accordingly, the invention further provides a package comprising a GRF molecule or the above-mentioned composition comprising a GRF molecule together with instructions to the effect that the GRF molecule or the above-mentioned composition comprising a GRF molecule is suitable for combination therapy with a protease inhibitor, i.e., that it may be administered to a subject undergoing treatment with or who is a candidate for treatment with a protease inhibitor. The kit or package may further comprise containers, buffers (e.g., to resuspend the compound(s), devices for administering the compound(s), etc.

“Suitable for combination therapy” in the present context refers to no or substantially no drug interactions between a GRF molecule and a protease inhibitor. In an embodiment, a GRF molecule and a protease inhibitor are suitable for combination therapy if the pharmacokinetics of the compounds is not significantly affected/modulated in the presence (relative to the absence) of the other compound. In an embodiment, a GRF molecule and a protease inhibitor are suitable for combination therapy if one or more pharmacokinetics parameters of the compounds is/are not affected/modulated by more than about 20% in the presence of the other compound (relative to the absence thereof). In an embodiment, one or more pharmacokinetic parameters of a compound are not “significantly affected” by the presence of another compound if they are not modulated by more than about 20% in the presence (relative to the absence) of the other compound. In an embodiment, the one or more pharmacokinetics parameters comprise the blood or plasma concentration of the compound. Methods for determining/analyzing pharmacokinetics parameters are well known in the art (see, for example, Thomas N. Tozer and Malcolm Rowland, Introduction to Pharmacokinetics and Pharmacodynamics: The Quantitative Basis of Drug Therapy, Lippincott Williams & Wilkins, 2006; Malcolm Rowland and Thomas N. Tozer, Clinical pharmacokinetics: concepts and applications, Williams & Wilkins, 3^{rd} edition, 1995). Pharmacokinetic analysis may be performed by noncompartmental (model independent) or compartmental methods. The amount of a compound in a biological fluid (blood, plasma urine) may be measured using well-known methods such as mass spectrometry. In an embodiment, a GRF molecule and a protease inhibitor are suitable for combination therapy if the maximal observed analyte concentration in plasma (Cₘₐₓ) and the area under the concentration-time curve (AUC) of either compound does not decrease by more than 20% or does not increase by more than 20% in the presence versus in the absence of the other compound of the pair. In another embodiment, a GRF molecule and a protease inhibitor are suitable for combination therapy if the ratio of (A) the AUC₀₋ₜ, AUC_{0-inf} and/or Cₘₐₓ values in presence of the other compound and (B) the AUC₀₋ₜ, AUC_{0-inf} and/or Cₘₐₓ values in the absence of the other compound is between about 0.85 to about 1.2 (i.e., about 85% to about 120%), for example between about 0.90 to about 1.1 (i.e. about 90% to about 110%). In another embodiment, a GRF molecule and a protease inhibitor are suitable for combination therapy if the 90% confidence intervals (CIs) for the ratios of (A) the AUC₀₋ₜ, AUC_{0-inf} and/or Cₘₐₓ values in presence of the other compound and (B) the AUC₀₋ₜ, AUC_{0-inf} and/or Cₘₐₓ values in the absence of the other compound is between about 0.75 to about 1.25 (i.e. about 75% to about 125%), for example between about 0.80 to about 1.25 (i.e., about 80% to about 125%), for example between about 0.90 to about 1.1 (i.e. about 90 to about 110%).

The present inventor has determined that a GRF molecule is suitable for combined therapy with a protease inhibitor, i.e., with no or substantially no drug interaction. Therefore, in an embodiment, the invention further provides a method comprising providing information (e.g., to a patient or a caregiver of the patient) that a GRF molecule and a protease inhibitor may be co-administered to the patient. In an embodiment, the method further comprises informing the patient or the caregiver of the patient that administration of the GRF molecule will have no or substantially no effect on the pharmacokinetics of the protease inhibitor, or that no modification of the treatment regimen (e.g., drug combination, dosage and/or frequency of administration) of the protease inhibitor is necessary.

The invention further provides a use of a GRF molecule for the above-noted combination therapy or to prevent or treat the above-noted conditions. The invention further provides a use of a GRF molecule for the preparation of a medicament for the above-noted combination therapy or to prevent or treat the above-noted conditions. The invention further provides a GRF molecule for use in the above-noted combination therapy or to prevent or treat the above-noted conditions.

In an embodiment the GRF molecule is a GRF analog, in a further embodiment (hexenoyl trans-3)hGRF(1-44)NH₂. In an embodiment, the above-mentioned GRF molecule (e.g., (hexenoyl trans-3)hGRF(1-44)NH₂) is administered at a daily dose of about 1 mg to about 2 mg, in a further embodiment at a daily dose of about 2 mg. In an embodiment, the GRF molecule (e.g., (hexenoyl trans-3)hGRF(1-44)NH₂) is administered subcutaneously.

In an embodiment, the above-mentioned method, use, (hexenoyl trans-3)hGRF(1-44)NH₂ or composition further comprises, prior to said treating (with a GRF molecule such as (hexenoyl trans-3)hGRF(1-44)NH₂), identifying or selecting a subject who is undergoing or who is a candidate for a treatment regimen with a protease inhibitor. The present inventors have determined that no or substantially no drug interaction following administration of a GRF molecule (hexenoyl trans-3)hGRF(1-44)NH₂) with a protease inhibitor, and thus that subjects in need of a treatment to increase GH levels (e.g., to treat conditions in which increasing GH levels is beneficial) but who are undergoing a protease inhibitor-based therapy (or who are a candidate for undergoing such therapy) may be identified or selected for treatment with a GRF molecule such as (hexenoyl trans-3)hGRF(1-44)NH₂.

As such, in an embodiment, there is also provided a method comprising:
identifying or selecting a subject who is (i) undergoing an antiretroviral protease inhibitor treatment regimen or is a candidate for an antiretroviral protease inhibitor treatment regimen, and (ii) in need of a treatment to increase GH levels (e.g., to treat conditions in which increasing GH levels is beneficial [e.g., a condition associated with fat accumulation or hypercholesterolemia, such as excess abdominal fat in an HIV-infected subject]);
administering to said subject a GRF molecule such as (hexenoyl trans-3)hGRF(1-44)NH₂.
wherein said method does not significantly affect pharmacokinetics or clearance of said protease inhibitor, and/or wherein said method does not require modifying said antiretroviral protease inhibitor treatment regimen.

The terms “subject” and “patient” are used interchangeably herein, and include a subject in need of the treatment described herein. In an embodiment, the subject is a mammal, in a further embodiment, a human.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”. The articles “a” and “an” are used herein to refer to one or to more than one (i.e., to at least one) of the grammatical object of the article. The term “such as” is used herein to mean, and is used interchangeably, with the phrase “such as but not limited to”. Throughout this application, various references are referred to describe more fully the state of the art to which this invention pertains. The disclosures of these references are hereby incorporated by reference into the present disclosure.

The following examples are illustrative of various aspects of the invention, and do not limit the broad aspects of the invention as disclosed herein.

### EXAMPLES

### Example 1

### Materials and Methods

### Study Drugs:

(hexenoyl trans-3)hGRF(1-44)NH₂: The GRF analog used in the studies described herein is (hexenoyl trans-3)hGRF(1-44)NH₂ (also referred to as [trans-3-hexenoyl]hGRF (1-44) amide and TH9507 herein), which is a synthetic human growth hormone releasing factor analog that comprises the 44-amino acid sequence of human growth hormone releasing factor (hGRF) on which a hexenoyl moiety, a C6 side chain has been anchored on Tyr 1 at the n-terminal. (hexenoyl trans-3)hGRF(1-44)NH₂ or Th9507 has the following structure:

(SEQ ID NO: 7)
(trans)CH₃—CH₂—CH═CH—CH₂—CO-Tyr-Ala-Asp-Ala-Ile-
Phe-Thr-Asn-Ser-Tyr-Arg-Lys-Val-Leu-Gly-Gln-Leu-
Ser-Ala-Arg-Lys-Leu-Leu-Gln-Asp-Ile-Met-Ser-Arg-
Gln-Gln-Gly-Glu-Ser-Asn-Gln-Glu-Arg-Gly-Ala-Arg-
Ala-Arg-Leu-NH₂.

(hexenoyl trans-3)hGRF(1-44)NH₂ is synthesized using FMOC solid phase peptide synthesis starting with Ramage Tricyclic Amide Resin. Protected amino acids and trans-3-hexenoyl acid are used for coupling whereby each protected amino acid and trans-3-hexenoyl acid is dissolved in DMF-treated with aluminum oxide with TBTU to assist in reducing racemization and DIPEA to promote activation before coupling. Completeness of couplings is monitored by the Kaiser ninhydrin test (E. Kaiser et al. Anal. Biochem. “Color Test for Detection of Free Terminal Amino Groups in the Solid Phase Synthesis of Peptides”) and the TNBS test (Means and Feeney, 1971, Holden-Day Inc. San Francisco “Chemical Modification of Proteins” p. 217).

The side chain protecting groups and the peptide-resin bond are cleaved by stirring the protected peptide-resin in a cleavage cocktail consisting of 90% TFA, 5% EDT and 5% water. The crude peptide is purified by HPLC through a three-stage purification scheme using the following buffers, 0.1% MSA, TEAP pH 6.5 and 2% HOAc affording pure [trans-3-hexenoyl]hGRF (1-44) amide (≧98.5%). The purified peptide lots are pooled and reconstituted in 0.5% acetic acid and lyophilized.

Lyophilization Process. The samples were lyophilized by freezing at −50° C. and holding, annealing to −10° C. and holding, primary drying at −10° C. under 100 mTorr and secondary drying at 25° C. under 100 mTorr.

2 ml of TH9507 (1 mg/ml injectable solution) was administered by subcutaneous injection under fasting conditions once daily for 7 consecutive days (daily dose of 2 mg TH9507).

Ritonavir: One Norvir® SEC 100 mg capsule (ritonavir) by Abbott Laboratories Ltd. Canada, under fed conditions, as indicated below.

### Subjects:

N=32 healthy adult (male and female) subjects were enrolled. Dosing occurred in two groups.

### Example 2

### Methods and Results

In a randomized, open-label, two-way crossover study, subjects were administered 2 mg TH9507 on days 1 to 7, with 100 mg ritonavir (N=32) co-administered on day 6 (Treatment A), and a single dose of ritonavir alone on day 6 (Treatment B) in a crossover manner. PK samples collected on day 6, measured ritonavir and TH9507 plasma concentrations. The A/B ratios and 90% confidence intervals (CI) within 80-125% would conclude that TH9507 has no clinically significant impact on ritonavir PKs. Administration of drugs and collection of samples was performed as indicated in Table I. Treatment A relates to administration of TH9507 and ritonavir and Treatment B relates to administration of ritonavir alone.

TABLE I
Administration of drugs and collection of samples

| | Treatment A | Treatment B |
|---|---|---|
| Days 1 to 5 | Administer TH9507 | — |
| | Administer TH9507; collect samples | — |
| | at 0, 0.1, 0.15, 0.2, 0.25, and 0.5 h | |
| Day 6 | Administer ritonavir; collect samples at 0, | |
| | 0.5, 1, 1.5, 2, 2.5, 3, 3.5, 4, 4.5, 5, 5.5, | |
| | 6, 6.5, 7, 8, 9, 10, 12, 16, 24, 30, 36, 48 h | |
| Day 7 | Administer TH9507 | — |

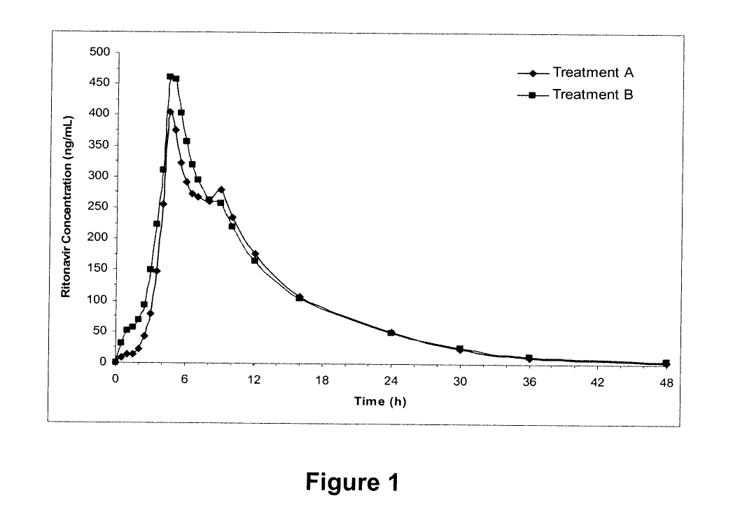

Bioanalytical: ELISA was used to measure plasma TH9507 and LC/MS/MS was used to measure plasma ritonavir. Concentration profiles of plasma ritonavir are shown in FIG. 1.

PK/statistics: PK parameters were calculated using standard noncompartmental approaches.

ANOVAs on natural log-transformed AUC₀₋ₜ, AUC_{0-inf} and Cₘₐₓ were conducted for ritonavir. The 90% CIs for the Treatment A/Treatment B LSM ratios for AUC₀₋ₜ. AUC_{0-inf} and Cₘₐₓ were to be within 80-125% in order to conclude that there is no clinically significant impact of TH9507 on ritonavir PK.

Blood sampling for TH9507 was designed to only estimate the PK profile; descriptive statistics were calculated for TH9507 plasma concentration data to demonstrate that the expected exposure was achieved.

TABLE 2
Summary of ANOVA results and PK parameters for Ritonavir

| | Treatment Means | | | 90% Confidence |
|---|---|---|---|---|
| Parameter | Treatment A | Treatment B | A/B Ratio (%) | Interval (%) |
| AUC₀₋ₜ (ng · h/mL) ^{a} | 3378.8 | 3722.6 | 90.8 | 83.8-98.3 |
| AUC_{0-inf} (ng · h/mL) ^{a} | 3465.3 | 3799.9 | 91.2 | 84.4-98.6 |
| Cₘₐₓ (ng/mL) ^{a} | 404.2 | 452.7 | 89.3 | 74.8-106.6 |
| Tₘₐₓ (h) ^{b} | 4.50 (4.50-16.00) | 4.50 (1.50-16.00) | — | — |
| T_{1/2} (h) ^{c} | 5.72 (23.0) | 6.26 (22.0) | — | — |

^{a} Geometric mean calculated by exponentiating the LSM from a model using log-transformed response;
^{b} Median (range);
^{c} Arithmetic mean (% CV)

TABLE 3
Summary of PK parameters for TH9507 in ritonavir studies

| Parameter | Mean | |
|---|---|---|
| AUC₀₋ₜ (pg · h/mL) ^{a} | 656.9 | (70.0) |
| AUC_{0-inf} (pg · h/mL) ^{a} | 790.4 | (47.1) |
| Cₘₐₓ (pg/mL) ^{a} | 2699.1 | (58.6) |
| Tₘₐₓ (h) ^{b} | 0.15 | (0.10-0.20) |
| T_{1/2} (h) ^{c} | 0.11 | (26.6) |

^{a} Geometric mean (% CV);
^{b} Median (range);
^{c} Arithmetic mean (% CV)

For ritonavir, the acceptance limits were met for AUC₀₋ₜ and AUC_{0-inf}; ratios and 90% CIs for AUCs were contained within the acceptance range. The lower 90% CI for Cₘₐₓ was 74.8%. Because the observed NB ratios for AUC₀₋ₜ, AUC_{0-inf} and Cₘₐₓ were approximately 90%, this is a minor decrease and no dose adjustment of ritonavir is required in the presence of TH9507.

These studies demonstrate that the impact of TH9507 on CYP3A activity is not significant. Therefore, ritonavir may be administered in conjunction with TH9507 without any change in their dosing regimen.

TABLE 4
Abbreviations used herein

| | |
|---|---|
| AE | Adverse Event |
| ANOVA | Analysis of Variance |
| ART | Antiretroviral therapy |
| AUC | Area under the concentration-time curve |
| AUC_{0-inf} | Area under the concentration-time curve from time |
| | zero to infinity (extrapolated) |
| AUC₀₋ₜ | Area under the concentration-time curve, from time |
| | zero to time of last non-zero concentration |
| CI | Confidence interval |
| Cₘₐₓ | Maximal observed analyte concentration in plasma |
| CPK | Creatine phosphokinase |
| CV | Coefficient of variation |
| CYP | Cytochrome P450 |
| CYP 3A | Cytochrome P450 3A |
| CYP 3A4 | Cytochrome P450 3A4 |
| DIPEA | N,N-diisopropylethylamine |
| DMF | N,N-dimethylformamide |
| EDT | 1,2-ethane dithiol |
| ELISA | Enzyme-linked immunosorbent assay |
| FMOC | 9-fluorenylmethyloxycarbonyl |
| (h)GH | (human) Growth Hormone |
| (h)GRF | (human) Growth Hormone-Releasing Factor |
| GHRH | Growth Hormone-Releasing Hormone |
| HIV | Human Immunodeficiency Virus |
| HPLC | High-performance liquid chromatography |
| LC/MS/MS | Liquid Chromatography/Mass Spectrometry/Mass |
| | Spectrometry |
| MSA | methane sulfonic acid |
| PK | Pharmacokinetic |
| SAE | Severe adverse event |
| SD | Standard Deviation |
| T_{1/2} | The apparent first-order terminal elimination half-life |
| TBTU | 2-(1H-benzotriazole-1-yl)-1,1,3,3-tetramethyluronium |
| | tetrafluoroborate |
| TEAP | triethylammonium phosphate |
| TFA | trifluoroacetic acid |
| Tₘₐₓ | Time of observed Cₘₐₓ |
| TNBS | trinitrobenzene sulfonic acid |

Although the present invention has been described hereinabove by way of specific embodiments thereof, it can be modified, without departing from the spirit and nature of the subject invention as defined in the appended claims.

1. A method of inducing GH levels in a subject undergoing an antiretroviral protease inhibitor treatment regimen or who is a candidate for an antiretroviral protease inhibitor treatment regimen, without modifying said treatment regimen, said method comprising administering to said subject an effective amount of (hexenoyl trans-3)hGRF(1-44)NH₂.

2. A method of treating excess abdominal fat in a HIV-infected subject with lipodystrophy, wherein said subject is undergoing an antiretroviral protease inhibitor treatment regimen or is a candidate for an antiretroviral protease inhibitor treatment regimen, without substantially modifying said treatment regimen, said method comprising administering to said subject an effective amount of (hexenoyl trans-3)hGRF(1-44)NH₂.

3. The method of claim 1, further comprising, prior to said treating, selecting a subject who is undergoing, or who is a candidate for, an antiretroviral protease inhibitor treatment regimen.

4. The method of claim 2, further comprising, prior to said treating, selecting a subject who is undergoing, or who is a candidate for, an antiretroviral protease inhibitor treatment regimen.

5. A method comprising providing information to a subject or to a caregiver of the subject that (hexenoyl trans-3)hGRF(1-44)NH₂ and a protease inhibitor can be co-administered to the subject.

6. The method of claim 5, further comprising providing information to the subject or to the caregiver of the subject that (hexenoyl trans-3)hGRF(1-44)NH₂ can be co-administered to the subject without affecting pharmacokinetics of said protease inhibitor.

7. The method of claim 5, further comprising providing information to the subject or to the caregiver of the subject that (hexenoyl trans-3)hGRF(1-44)NH₂ can be co-administered to the subject without modifying the treatment regimen of said protease inhibitor.

8. The method of claim 5, further comprising selecting a subject who is undergoing or who is a candidate for a treatment regimen with (hexenoyl trans-3)hGRF(1-44)NH₂ and with a protease inhibitor.

9. The method of claim 1, wherein the protease inhibitor is ritonavir.

10. The method of claim 1, wherein the (hexenoyl trans-3)hGRF(1-44)NH₂ is administered at a daily dose of about 2 mg.

11. The method of claim 1, wherein the (hexenoyl trans-3)hGRF(1-44)NH₂ is administered subcutaneously.

12. The method of claim 2, wherein the protease inhibitor is ritonavir.

13. The method of claim 2, wherein the (hexenoyl trans-3)hGRF(1-44)NH₂ is administered at a daily dose of about 2 mg.

14. The method of claim 2, wherein the (hexenoyl trans-3)hGRF(1-44)NH₂ is administered subcutaneously.

15. The method of claim 5, wherein the protease inhibitor is ritonavir.

16. The method of claim 5, wherein the (hexenoyl trans-3)hGRF(1-44)NH₂ is administered at a daily dose of about 2 mg.

17. The method of claim 5, wherein the (hexenoyl trans-3)hGRF(1-44)NH₂ is administered subcutaneously.


SEQUENCE LISTING (7 sequences)

SEQ ID NO: 1 - protein, 44 aa, Artificial sequence
  GRF peptide
XXDAIFTXSYRKXLXQLXARKLLXXIXXRXXXXXXXXXXXXXXX

SEQ ID NO: 2 - protein, 44 aa, Homo sapiens
  MISC_FEATURE (44)..(44): Leu residue is capped with an unsubstituted amide moiety
YADAIFTNSYRKVLGQLSARKLLQDIMSRQQGESNQERGARARL

SEQ ID NO: 3 - protein, 44 aa, Artificial sequence; identical to SEQ ID NO: 2
  Amino acid sequence of human GRF

SEQ ID NO: 4 - protein, 29 aa, Homo sapiens
  MISC_FEATURE (29)..(29): Arg residue is capped with an unsubstituted amide moiety
YADAIFTNSYRKVLGQLSARKLLQDIMSR

SEQ ID NO: 5 - protein, 29 aa, Artificial sequence
  Amino acid sequence of minimum active core of human GRF
YADAIFTNSYRKVLGQLSARKLLQDIMSR

SEQ ID NO: 6 - protein, 15 aa, Artificial sequence
  Amino acid sequence corresponding to positions 30 to 44 of human GRF
QQGESNQERGARARL

SEQ ID NO: 7 - protein, 44 aa, Artificial sequence; identical to SEQ ID NO: 2
  Modified GRF peptide